(19) United States
(12) Patent Application Publication
(10) Pub. No.: US 2026/0306140 A1
(43) Pub. Date: Oct. 8, 2026

(54) SYSTEM AND METHOD FOR FIXATION OF IMPLANTABLE SENSORS

(71) Applicant: THE JOHNS HOPKINS UNIVERSITY, Baltimore, MD (US)

(72) Inventors: Amir MANBACHI, Baltimore, MD (US); Nicholas THEODORE, Ruxton, MD (US); Constantin SMIT, Baltimore, MD (US); Kayla ROBINSON, Baltimore, MD (US); Kelley KEMPSKI LEADINGHAM, Baltimore, MD (US)

(21) Appl. No.: 19/481,454

(22) PCT Filed: Mar. 21, 2024
(86) PCT No.: PCT/US2024/020941

§ 371 Date: Nov. 4, 2025

Related U.S. Application Data
(60) Provisional application No. 63/501,175, filed on May 10, 2023.

(51) Int. Cl. A61B 17/70; A61B 8/00; A61B 8/06; A61B 17/00

(52) U.S. Cl. CPC A61B 17/7032; A61B 8/06; A61B 8/4209; A61B 8/4444; A61B 17/7035; A61B 2017/00106

(57) ABSTRACT

An implantable apparatus includes one or more screws configured to be screwed into one or more bones. The apparatus also includes a rod configured to extend through the one or more screws. The apparatus also includes a support structure configured to have the rod extend therethrough. The support structure is configured to support a sensor such that the sensor is positioned at least partially over an internal object to be imaged. The support structure is configured to adjust a position of the sensor in a first direction, a second direction, or both. The first and second directions are perpendicular to one another.

### CROSS-REFERENCE TO RELATED APPLICATIONS

This application is the national stage entry of International Patent Application No. PCT/US2024/020941, filed on Mar. 21, 2024, and published as WO 2024/233002 A1 on Nov. 14, 2024, which claims the benefit of U.S. Provisional Patent Application No. 63/501,175, filed on May 10, 2023, which are hereby incorporated by reference in their entireties.

### GOVERNMENT SUPPORT

This invention was made with government support under cooperative agreement no. N66001-20-2-4075, awarded by the U.S. Department of the Navy. The government has certain rights in the invention.

### FIELD OF THE DISCLOSURE

The present disclosure relates generally to systems and methods for implanting sensors. More particularly, the present disclosure relates to systems and methods for implanting and adjusting an implantable sensor in a patient during a surgical procedure.

### BACKGROUND OF THE DISCLOSURE

Implantable sensors may be implanted into a patient during a surgical procedure. They have been used in medical research for measuring parameters such as force, torque, pressure, and temperature inside the human body. However, conventional implantable sensors cannot be adjusted laterally and/or vertically within the patient.

### SUMMARY

An implantable apparatus includes one or more screws configured to be screwed into one or more bones. The apparatus also includes a rod configured to extend through the one or more screws. The apparatus also includes a support structure configured to have the rod extend therethrough. The support structure is configured to support a sensor such that the sensor is positioned at least partially over an internal object to be imaged. The support structure is configured to adjust a position of the sensor in a first direction, a second direction, or both. The first and second directions are perpendicular to one another.

An implantable apparatus that is configured to be implanted in a patient is also disclosed. The apparatus includes one or more first screws configured to be screwed into one or more bones on a first side of an internal object to be imaged. The apparatus also includes one or more second screws configured to be screwed into the one or more bones on a second side of the internal object to be imaged. The one or more first screws and the one or more second screws are pedicle screws. The one or more bones are vertebra. The internal object to be imaged is a spinal cord. The apparatus also includes a first rod configured to extend through the one or more first screws. he apparatus also includes a second rod configured to extend through the one or more second screws. The internal object to be imaged is configured to be positioned between the first and second rods. The apparatus also includes a first support structure configured to have the first rod extend therethrough. The first support structure is configured to support a sensor proximate to a first end of the sensor. The sensor is an implantable ultrasound sensor. The apparatus also includes a second support structure configured to have the second rod extend therethrough. The second support structure is configured to support the sensor proximate to a second end of the sensor. The first and second support structures are configured to support the sensor such that the sensor is at least partially positioned over the internal object to be imaged. The first and second support structures each include a rod portion and a sensor portion. The rod portion is configured to receive the first rod or the second rod therethrough. The rod portion is configured adjust a position of the sensor in a first direction. The rod portion includes a plurality of posts that define a first recess. The first rod or the second rod is configured to be received into the first recess. The rod portion also includes one or more actuators positioned at least partially between the posts. The one or more actuators are engaged with internal threads defined on inner surfaces of the posts. The one or more actuators are configured to rotate to move in the first direction. The sensor portion is configured to be coupled to the rod portion and to support the sensor. The sensor portion is configured to be rotated with respect to the rod portion. The sensor portion is configured to adjust a position of the sensor in a second direction that is perpendicular to the first direction. The sensor portion includes a shaft that extends at least partially through the rod portion. The sensor portion also includes a clamp coupled to the shaft. The clamp supports the sensor therein.

A method is also disclosed. The method includes screwing one or more screws into one or more bones in a patient during a surgical procedure. The method also includes inserting a rod through the one or more screws. The method also includes positioning a support structure such that the rod extends through the support structure. The method also includes inserting a sensor at least partially into the support structure. The method also includes adjusting the support structure to vary a position of the sensor in a first direction, a second direction, or both. The first and second directions are perpendicular to one another.

### BRIEF DESCRIPTION OF THE FIGURES

FIG. 1 illustrates a top view of an implantable apparatus for holding an implantable sensor, according to an embodiment.

FIG. 2 illustrates an end view of the implantable apparatus, according to an embodiment.

FIG. 3 illustrates a side view of a support structure of the implantable apparatus that includes a rod portion coupled to a sensor portion, according to an embodiment.

FIG. 4 illustrates a side view of the rod portion coupled to the sensor portion with the sensor portion rotated with respect to the rod portion, according to an embodiment.

FIG. 5 illustrates a side view of the rod portion, according to an embodiment.

FIG. 6 illustrates a side view of the sensor portion of the implantable apparatus, according to an embodiment.

FIG. 7 illustrates a top view of the sensor portion, according to an embodiment.

FIG. 8 illustrates a flowchart of a method for installing and/or adjusting the implantable apparatus, according to an embodiment.

### DETAILED DESCRIPTION

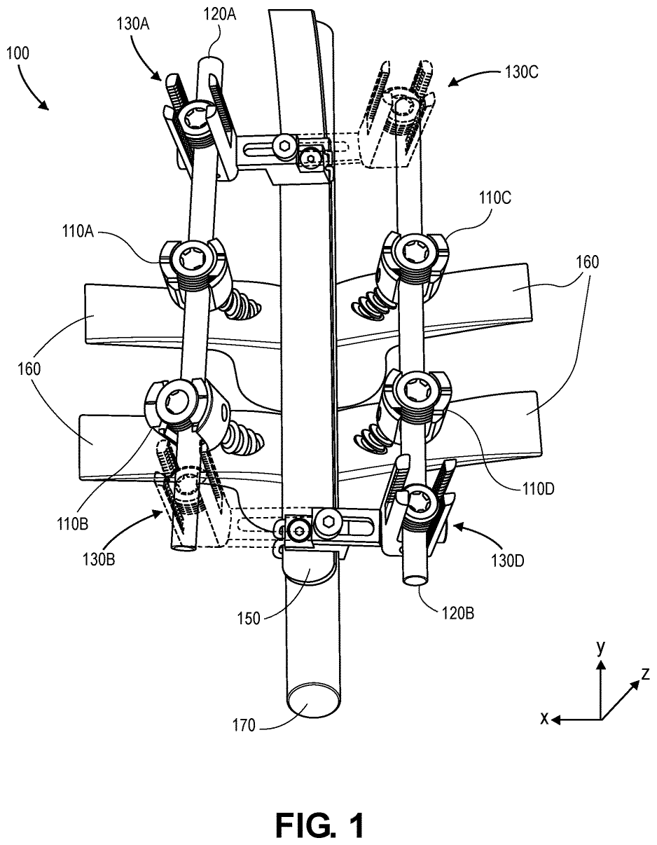

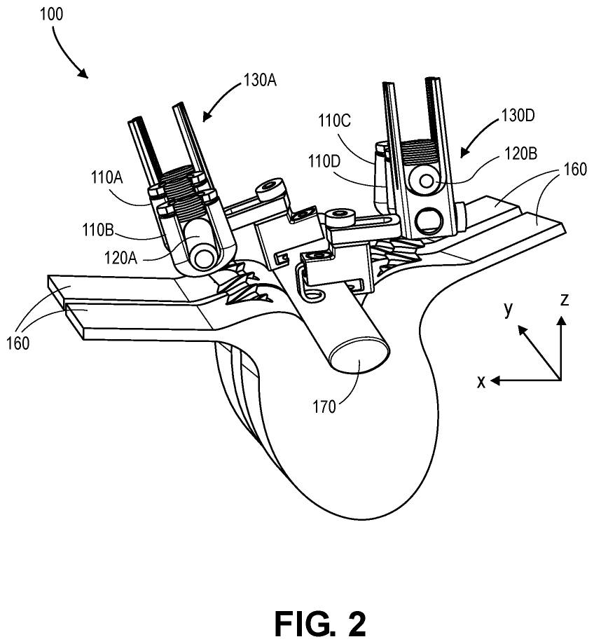

FIGS. 1 and 2 illustrate a top view and an end view of an implantable apparatus 100 for holding an implantable sensor 150, according to an embodiment. As described in greater detail below, the implantable apparatus 100 may be configured to adjust the position of the sensor 150 laterally and/or vertically within a patient. The implantable apparatus 100 may also or instead be configured to adjust the orientation of (e.g., rotate) the sensor 150 within the patient. The ability to adjust the position and/or orientation of the sensor 150 may allow the implantable apparatus 100 and the sensor 150 to be used in different patients having different anatomical structures. It may also allow the position and/or orientation of the sensor 150 to be adjusted in a single patient over time.

The implantable apparatus 100 may include one or more screws (four are shown: 110A-110D). The screws 110A-110D may be configured to be screwed into one or more bones 160 in the patient. In the example shown, the screws 110A-110D may be pedicle screws, and the bones 160 may be vertebra. One or more (e.g., two) of the screws 110A, 110B may be positioned on a first side of an internal object to be imaged 170, and one or more (e.g., two) of the screws 110C, 110D may be positioned on a second (e.g., opposite) side of the internal object to be imaged 170. In the example shown, the internal object to be imaged 170 is a spinal cord.

The implantable apparatus 100 may also include one or more rods (two are shown: 120A, 120B). The first rod 120A may be configured to be coupled to (e.g., inserted through) the screws 110A, 110B, and the second rod 120B may be configured to be coupled to (e.g., inserted through) the screws 110C, 110D. Thus, the rods 120A, 120B may be positioned on opposite sides of the internal object to be imaged 170. In the example shown, the rods 120A, 120B are straight; however, in other embodiments, one or both of the rods 120A, 120B may be curved depending upon the anatomy of the patient and/or the positioning of the components through which the rods 120A, 120B are to extend.

The implantable apparatus 100 may also include one or more support structures (four are shown: 130A-130D). The support structures 130A-130D may not be (e.g., directly) coupled to the patient. For example, the support structures 130A-130D may not be (e.g., directly) screwed into the one or more bones 160. Rather, the support structures 130A, 130B may be configured to be coupled to the rod 120A, and the support structures 130C, 130D may be configured to be coupled to the rod 120B. More particularly, the support structures 130A, 130B may be configured to have the rod 120A extend therethrough, and the support structures 130C, 130D may be configured to have the rod 120B extend therethrough. Thus, the support structures 130A, 130B may be positioned on opposite sides of the internal object to be imaged 170 from the support structures 130C, 130D. In one embodiment, one or more of the support structures (e.g., 130B and 130C) may be omitted to reduce the number of components implanted into the patient. Thus, the support structures 130B, 130C are shown in dashed lines in FIG. 1 and omitted in FIG. 2.

The support structures 130A-130D may be configured to support the sensor 150. More particularly, the support structures 130A, 130C may be configured to support the sensor 150 proximate to a first (e.g., axial) end thereof, and the support structures 130B, 130D may be configured to support the sensor 150 proximate to a second (e.g., axial) end thereof. The sensor 150 may be supported such that it is positioned between the screws 110A, 110B and the screws 110C, 110D, between the rods 120A, 120B, between the support structures 130A, 130B and the support structures 130C, 130D, and is at least partially positioned over (e.g., parallel with) the internal object to be imaged 170.

In the example shown, the sensor 150 may be or include a multi-function spinal cord implant (MUSIC) sensor, which provides temporary ultrasound imaging during the acute recovery phase after a spinal cord injury (SCI). Thus, the sensor 150 may be or include an epidural, flexible, wired patch placed over the site of injury after surgical decompression of the spinal cord 170. The sensor 150 may perform ultrasonic imaging of spinal cord 170 and Doppler blood flow imaging to help clinicians directly quantify blood flow and identify perfusion ranges for optimal patient recovery. Furthermore, the ultrasound images generated by the sensor 150 can monitor the spinal cord 170 for swelling and development of hematoma.

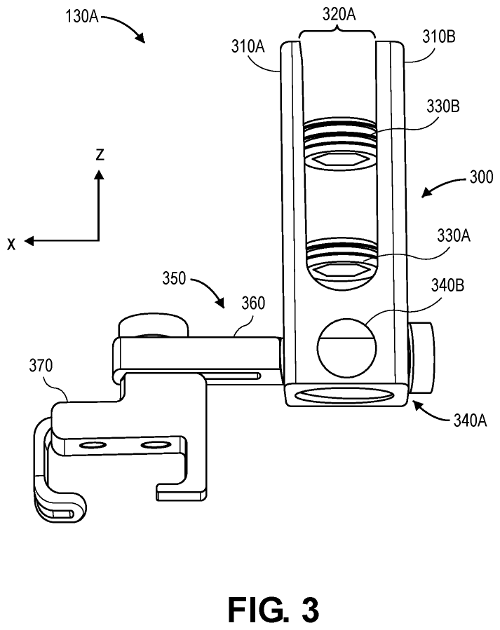

FIG. 3 illustrates a side view of one of the support structures 130A of the implantable apparatus 100, according to an embodiment. The support structure 130A may include a first (e.g., rod) portion 300 and a second (e.g., sensor) portion 350. The rod portion 300 and the sensor portion 350 may be substantially perpendicular to one another. The rod portion 300 may extend in the Z direction, and the sensor portion 350 may extend in the X direction.

The rod portion 300 may be configured to receive the rod 120A therethrough. More particularly, the rod portion 300 may include one or more (e.g., vertical) posts (two are shown: 310A, 310B) that are parallel to the Z axis. The posts 310A, 310B may define a first recess (or opening) 320A. The rod 120A is configured to extend through the first recess 320A in a direction that is parallel to the Y axis (into and out of the Figure).

The rod portion 300 may also include one or more first openings 340A that are configured to have at least a portion of the sensor portion 350 extend therethrough. The one or more first openings 340A may be positioned below the rod 120A and/or the first recess 320A (e.g., along the Z axis).

The sensor portion 350 may include a shaft 360. As mentioned above, the shaft 360 may be configured to extend through the one or more first openings 340A. The sensor portion 350 may also include a clamp 370 that is coupled to the shaft 360. The sensor 150 may extend through and/or be supported by the clamp 370.

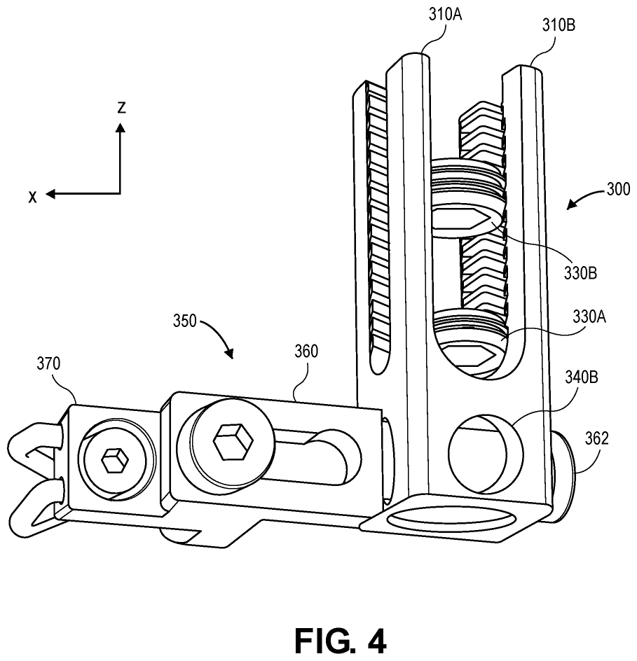

FIG. 4 illustrates a side view of the rod portion 300 coupled to the sensor portion 350 with the sensor portion 350 rotated with respect to the rod portion 300, according to an embodiment. More particularly, the sensor portion 350 (e.g., the shaft 360) may include a first actuator 362, which may be loosened to allow the shaft 360 to be rotated (e.g., around the X axis). This may be beneficial when collecting Doppler blood flow images to increase the received signal by changing the angle in Doppler frequency shift. This may also or instead be beneficial to position the sensor 150 at an angle or perpendicular to the internal object to be imaged 170, which may not itself be straight (e.g., the spine has peaks and valleys).

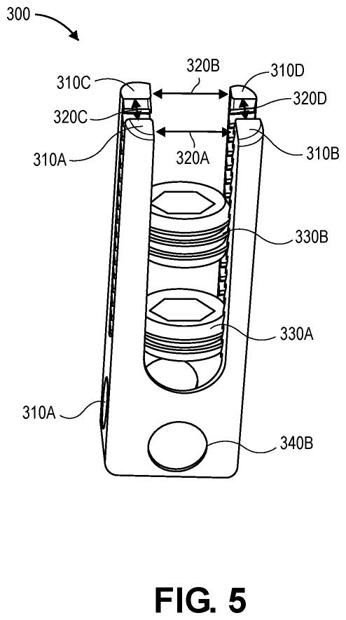

FIG. 5 illustrates a side view of the rod portion 300, according to an embodiment. In one embodiment, the rod portion 300 may include four posts 310A-310D. The posts 310A, 310B may define a first recess 320A. The posts 310C, 310D may define a second recess 320B that is aligned with the first recess 320A. The posts 310A, 310C may define a third recess 320C. The posts 310B, 310D may define a fourth recess 320D that is aligned with the third recess 320C. The recesses 320A, 320B may be perpendicular to the recesses 320C, 320D. The recesses 320A, 320B may have a first width 322, and the recesses 320C, 320D may have a second (e.g., different) width (not shown). The different widths may be used to accommodate rods of different sizes (e.g., diameters). For example, the rod 120A may extend through the recesses 320A, 320B. However, if a different rod is instead used that has a different size, the rod portion 300 may be rotated (e.g., by 90 degrees), and the different rod may extend through the recesses 320C, 320D. Accordingly, the rod portion 300 may also include one or more second openings 340B that are rotationally offset from the first openings 340A (e.g., by 90 degrees). The second openings 340B are configured to receive the shaft 360 if the rod portion 300 is rotated (e.g., by 90 degrees).

The rod portion 300 may be configured to adjust the height of the rod 120A, and thus the height of the sensor 150 (e.g., along the Z axis) with respect to the internal object to be imaged 170. For example, an inner surface of the rod portion 300 (e.g., the posts 310A-310D) may include internal threads. One or more actuators (two are shown: 330A, 330B) may be positioned at least partially within the rod portion 300 and engaged with the internal threads. In the example shown, the actuators 330A, 330B are nuts. The rod 120A may extend through the recesses 320A, 320B (or recesses 320C, 320D) and be positioned between the nuts 330A, 330B. The nuts 330A, 330B may be rotated to adjust their vertical positioning (e.g., along the Z axis), which may be used to adjust the vertical positioning of the rod 120A and the sensor 150. In another embodiment, one or more of the nuts may be omitted, and the actuator(s) 330A, 330B may instead include one or more biasing members (e.g., springs) that are configured to adjust the position of the rod 120A along the Z axis, thereby adjusting the position of the sensor 150 along the Z axis.

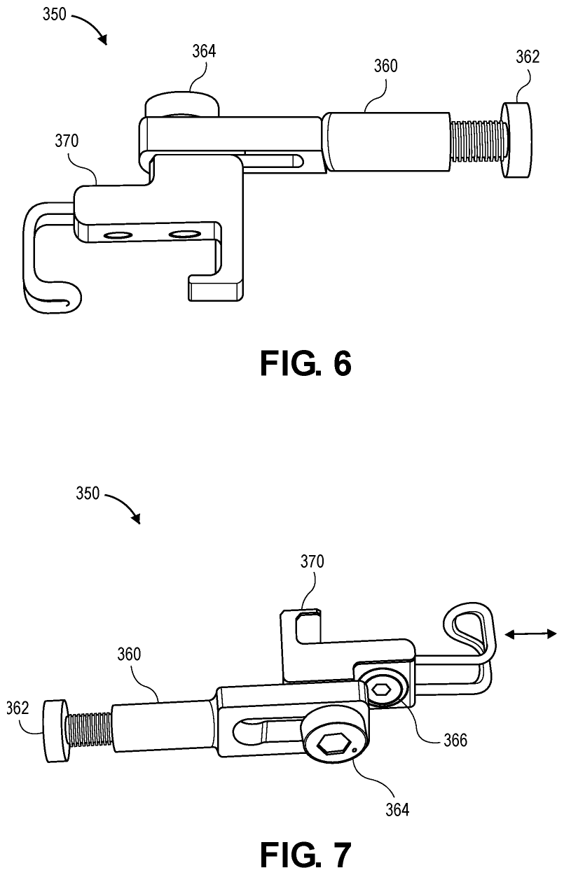

FIG. 6 illustrates a side view of the sensor portion 350 of the implantable apparatus 100, according to an embodiment. As mentioned above, the shaft 360 may include the first actuator (e.g., screw) 362, which is located proximate to a first axial end of the shaft 360 that is distal from the clamp 370. The first actuator 362 may be loosened and/or removed to allow the sensor portion 350 to be rotated with respect to the rod portion 350. The first actuator 362 may also be re-inserted and/or tightened to secure the shaft 360 to the rod portion 300 and to secure the rotational position of the sensor portion 350 with respect to the rod portion 300.

The sensor portion 350 may also include a second actuator (e.g., screw) 364 that is proximate to a second axial end of the shaft 360 that is closer to the clamp 370. The second actuator 364 may be loosened and/or removed to allow the position of the clamp 370 to be adjusted with respect to the shaft 360 (e.g., along the X axis). This may be used to position the sensor 150 over the internal object to be imaged 170. The second actuator 364 may also be re-inserted and/or tightened to secure the position of the clamp 370.

FIG. 7 illustrates a top view of the sensor portion 350, according to an embodiment. The clamp 370 may include a third actuator (e.g., screw) 366. The third actuator 366 may be loosened and/or removed to extend (e.g., open) a portion of the clamp 370 (e.g., along the X axis). The sensor 150 may be inserted into or removed from the clamp 370 when the clamp 370 is in the open position. Once the sensor 150 is inserted, the portion of the clamp 370 may be moved in the opposite direction and into contact with the sensor 150. The third actuator 366 may then be re-inserted and/or tightened to secure the sensor 150 within the clamp 370. In another embodiment, the third actuator 366 may be omitted, and the clamp 370 may not be extended and retracted between the open and closed positions. Rather, the sensor 150 may be a friction fit within the clamp 370. Although the actuators 364, 366 are shown positioned over the sensor 150 and the internal object to be imaged 170 (see FIG. 1), in another embodiment, the actuator 364 and/or the actuator 366 may be offset (e.g., along the X axis) so that they are not positioned over the sensor 150 and/or the internal object to be imaged 170. This may mitigate the risk of the actuators 364, 366 falling into the internal cavity of the patient and/or damaging the internal object to be imaged 170.

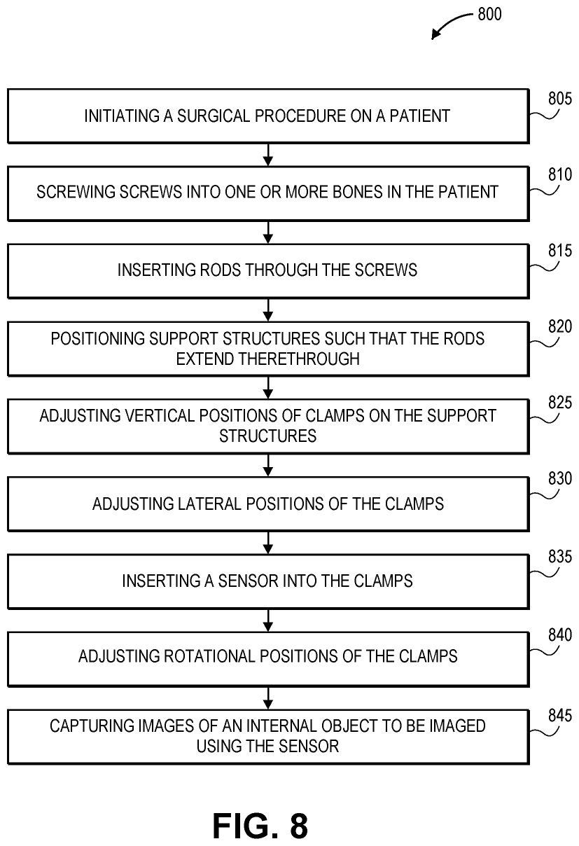

FIG. 8 illustrates a flowchart of a method 800 for installing and/or adjusting the implantable apparatus 100, according to an embodiment. An illustrative order of the method 800 is described below; however, one or more steps of the method 800 may be performed in a different order, simultaneously, repeated, or omitted.

The method 800 may include initiating a surgical procedure on the patient, as at 805. This may include creating an incision in the patient to access the one or more bones 160 and/or the internal object to be imaged 170. As mentioned above, in one example, the bones 160 may be vertebra, and the internal object to be imaged 170 may be a spinal cord.

The method 800 may also include screwing the screws 110A-110D into the one or more bones 160, as at 810. As mentioned above, the screws 110A, 110B may be positioned on a first side of the internal object to be imaged 170, and the screws 110C, 110D may be positioned on a second (e.g., different) side of the internal object to be imaged 170.

The method 800 may also include inserting the rods 120A, 120B through the screws 110A-110D, as at 815. More particularly, this may include inserting the rod 120A through the screws 110A, 110B, and inserting the rod 120B through the screws 110C and 110D. Thus, the rods 120A, 120B may be positioned on opposite sides of the internal object to be imaged 170.

The method 800 may also include positioning the support structures 130A-130D such that the rods 120A, 120B extend therethrough, as at 820. The support structures 130A, 130B may be positioned on the first side of the internal object to be imaged 170, and the support structures 130C, 130D may be positioned on the second side of the internal object to be imaged 170. As mentioned above, the support structures 130B, 130C may be omitted.

The rods 120A, 120B may be inserted into or through the recesses 320A, 320B (or 320C, 320D) in the rod portions 300 of the support structures 130A-130D. In one embodiment, the rods 120A, 120B may be inserted laterally (e.g., in a direction parallel to the Y axis) between the actuators (e.g., screws) 330A, 330B. In another embodiment, the upper actuators 330B may not be present when the rods 120A, 120B are inserted, and the rods 120A, 120B may be inserted vertically (e.g., in a direction parallel to the Z axis) down onto the lower actuators 330A. Once the rods 120A, 120B are inserted, the upper actuators 330B may be screwed down into contact with the rods 120A, 120B.

Before or after the support structures 130A-130D are positioned, it may be determined that the (e.g., vertical) positions of the clamps 370 may be too high or too low (e.g., along the Z axis) with respect to the internal object to be imaged 170. In this case, the method 800 may include adjusting vertical positions of the support structures 130A-130D, as at 825. This may include rotating the actuators 330A, 330B to raise or lower them within the posts 310A-310D of the rod portions 300. Raising the actuators 330A, 330B may lower the clamps 370 toward the internal object to be imaged 170. Conversely, lowering the actuators 330A, 330B may raise the clamps 370 away from the internal object to be imaged 170.

To accomplish this, the rods 120A, 120B and the upper actuators 330B and may be removed from the rod portions 300. Then, the lower actuators 330A may be rotated to raise or lower them within the rod portions 300. In one embodiment, once the lower actuators 330A are in the desired positions, the upper actuators 330B may be inserted and rotated to lower them toward the lower actuators 330B. Then, the rods 120A, 120B may be re-inserted laterally between the actuators 330A, 330B, and the upper actuators 330B may be lowered into contact with the rods 120A, 120B. In another embodiment, once the lower actuators 330A are in the desired positions, the rods 120A, 120B may be re-inserted vertically downward through the recesses 320A (or 320B) down into contact with the lower actuators 330A. The upper actuators 330B may then be inserted and lowered into contact with the rods 120A, 120B.

Before or after the support structures 130A-130D are positioned, it may also be determined that the (e.g., lateral) positions of the clamps 370 may be misaligned (e.g., along the X axis) with the internal object to be imaged 170. In this case, the method 800 may include adjusting lateral positions of the clamps 370, as at 830. This may include rotating the actuators 364 to loosen or remove them, and then moving the clamps 370 along the X axis with respect to the shaft 360. The clamps 370 may be moved toward or away from the rod portions 300 (e.g., until the clamps 370 are positioned over the internal object to be imaged 170). The actuators 364 may then be re-inserted and rotated to tighten the actuators 364, thereby securing the positions of the clamps 370 with respect to the shafts 360 along the X axis. In one embodiment, this may cause the rod portions 300 to become misaligned with the screws 110A-110D. When this occurs, the rods 120A, 120B may be bent (or replaced with bent rods) so that they will extend through the screws 110A-110D and rod portions 300.

The method 800 may also include inserting the sensor 150 into the clamps 370 of the support structures 130A-130D, as at 835. In one embodiment, this may include rotating the actuators 366 to loosen or remove them, and then extending (e.g., opening) the clamps 370 (e.g., along the X axis). The sensor 150 may be inserted into (or removed from) the clamps 370 when the clamps 370 are in the open positions. The clamps 370 may then be retracted into the closed positions, and the actuators 366 may then be re-inserted and/or tightened to secure the sensor 150 therein. As mentioned above, in another embodiment, the clamps 370 may not open and close, and the sensor 150 may be inserted and secured therein via a friction fit.

Before or after the sensor 150 is inserted, it may be determined that at least a portion of the sensor 150 should be bent or rotated. In this case, the method 800 may also include adjusting rotational positions (e.g., orientations) of the clamps 370, as at 840. This may include rotating the actuators 362 to loosen or remove them, and then rotating the sensor portion 350 around the X axis with respect to the rod portion 350, as shown in FIG. 4.

Once the implantable apparatus 100 (and the sensor 150) are in place, the method 800 may also include capturing images of the internal object to be imaged 170 using the sensor 150, as at 845. The images may be ultrasound images and/or Doppler blood flow images.

As used herein, the terms “inner” and “outer”; “up” and “down”; “upper” and “lower”; “upward” and “downward”; “upstream” and “downstream”; “above” and “below”; “inward” and “outward”; and other like terms as used herein refer to relative positions to one another and are not intended to denote a particular direction or spatial orientation. The terms “couple,” “coupled,” “connect,” “connection,” “connected,” “in connection with,” and “connecting” refer to “in direct connection with” or “in connection with via one or more intermediate elements or members.”

The foregoing description, for purposes of explanation, used specific nomenclature to provide a thorough understanding of the disclosure. However, it will be apparent to one skilled in the art that the specific details are not required in order to practice the systems and methods described herein. The foregoing descriptions of specific examples are presented for purposes of illustration and description. They are not intended to be exhaustive of or to limit this disclosure to the precise forms described. Many modifications and variations are possible in view of the above teachings. The examples are shown and described in order to best explain the principles of this disclosure and practical applications, to thereby enable others skilled in the art to best utilize this disclosure and various examples with various modifications as are suited to the particular use contemplated. It is intended that the scope of this disclosure be defined by the claims and their equivalents below.

1. An implantable apparatus, comprising:
one or more screws configured to be screwed into one or more bones;
a rod configured to extend through the one or more screws; and
a support structure configured to have the rod extend therethrough, wherein the support structure is configured to support a sensor such that the sensor is positioned at least partially over an internal object to be imaged, wherein the support structure is configured to adjust a position of the sensor in a first direction, a second direction, or both, and wherein the first and second directions are perpendicular to one another.

2. The implantable apparatus of claim 1, wherein the one or more screws comprise pedicle screws, wherein the one or more bones comprise vertebra, wherein the internal object to be imaged comprises a spinal cord, and wherein the sensor comprises an implantable ultrasound sensor.

3. The implantable apparatus of claim 1, wherein the support structure comprises a rod portion configured to receive the rod therethrough, and wherein the rod portion is configured adjust the position of the rod in the first direction.

4. The implantable apparatus of claim 3, wherein the rod portion comprises a plurality of posts that define a first recess, wherein the rod is configured to be received into the first recess.

5. The implantable apparatus of claim 3, wherein the rod portion comprises one or more actuators that are configured to rotate to move in the first direction to adjust a position of the rod, thereby adjusting the position of the sensor in the first direction.

6. The implantable apparatus of claim 1, wherein the support structure comprises a sensor portion configured to support the sensor, and wherein the sensor portion is configured to adjust the position of the sensor in the second direction.

7. The implantable apparatus of claim 6, wherein the sensor portion comprises an actuator that is configured to allow the sensor portion to be rotated around an axis that is parallel to the second direction.

8. The implantable apparatus of claim 6, wherein the sensor portion comprises:
a clamp that is configured to support the sensor therein; and
an actuator that is configured to allow a position of the clamp to be adjusted in the second direction, thereby adjusting the position of the sensor in the second direction.

9. The implantable apparatus of claim 6, wherein the sensor portion comprises:
a clamp that is configured to support the sensor therein; and
an actuator that is configured to allow a portion of the clamp to extend and retract in the second direction.

10. An implantable apparatus that is configured to be implanted in a patient, the implantable apparatus comprising:
one or more first screws configured to be screwed into one or more bones on a first side of an internal object to be imaged;
one or more second screws configured to be screwed into the one or more bones on a second side of the internal object to be imaged, wherein the one or more first screws and the one or more second screws comprise pedicle screws, wherein the one or more bones comprise vertebra, and wherein the internal object to be imaged comprises a spinal cord;
a first rod configured to extend through the one or more first screws;
a second rod configured to extend through the one or more second screws, wherein the internal object to be imaged is configured to be positioned between the first and second rods;
a first support structure configured to have the first rod extend therethrough, wherein the first support structure is configured to support a sensor proximate to a first end of the sensor, and wherein the sensor comprises an implantable ultrasound sensor;
a second support structure configured to have the second rod extend therethrough, wherein the second support structure is configured to support the sensor proximate to a second end of the sensor, wherein the first and second support structures are configured to support the sensor such that the sensor is at least partially positioned over the internal object to be imaged, and wherein the first and second support structures each comprise:
a rod portion configured to receive the first rod or the second rod therethrough, wherein the rod portion is configured adjust a position of the sensor in a first direction, and wherein the rod portion comprises:
a plurality of posts that define a first recess, wherein the first rod or the second rod is configured to be received into the first recess; and
one or more actuators positioned at least partially between the posts, wherein the one or more actuators are engaged with internal threads defined on inner surfaces of the posts, and wherein the one or more actuators are configured to rotate to move in the first direction; and
a sensor portion configured to be coupled to the rod portion and to support the sensor, wherein the sensor portion is configured to be rotated with respect to the rod portion, wherein the sensor portion is configured to adjust a position of the sensor in a second direction that is perpendicular to the first direction, and wherein the sensor portion comprises:
a shaft that extends at least partially through the rod portion; and
a clamp coupled to the shaft, wherein the clamp supports the sensor therein.

11. The implantable apparatus of claim 10, wherein the posts also define a second recess, wherein the first and second recesses are perpendicular to one another, and wherein the first and second recesses have different widths.

12. The implantable apparatus of claim 10, wherein the one or more actuators comprise a first actuator and a second actuator, and wherein the first rod or the second rod is configured to be positioned between the first and second actuators.

13. The implantable apparatus of claim 10, wherein the sensor portion further comprises a first actuator that is configured to allow the sensor portion to be rotated with respect to the rod portion.

14. The implantable apparatus of claim 13, wherein the sensor portion further comprises a second actuator that is configured to allow a position of the clamp to be adjusted in the second direction.

15. The implantable apparatus of claim 14, wherein the sensor portion further comprises a third actuator that is configured to allow a first portion of the clamp to extend and retract in the second direction with respect to a second portion of the clamp.

16. A method, comprising:
screwing one or more screws into one or more bones in a patient during a surgical procedure;
inserting a rod through the one or more screws;
positioning a support structure such that the rod extends through the support structure;
inserting a sensor at least partially into the support structure; and
adjusting the support structure to vary a position of the sensor in a first direction, a second direction, or both, wherein the first and second directions are perpendicular to one another.

17. The method of claim 16, wherein the support structure is not directly connected to the one or more bones or any other part of the patient.

18. The method of claim 16, wherein the support structure is adjusted to vary the position of the sensor in the first direction by rotating one or more actuators of the support structure to vary a position of the one or more actuators in the first direction, thereby varying a position of the rod in the first direction and varying the position of the sensor in the first direction.

19. The method of claim 16, wherein the support structure is adjusted to vary the position of the sensor in the second direction by rotating an actuator and then moving a first portion of the support structure in the second direction with respect to a second portion of the support structure.

20. The method of claim 16, further comprising adjusting the support structure to vary an orientation of the sensor by rotating a first portion of the support structure with respect to a second portion of the support structure.
